(19) United States
(12) Patent Application Publication
(10) Pub. No.: US 2004/0241147 A1
(43) Pub. Date: Dec. 2, 2004

(54) New isolated dendritic cells, a process for preparing the same and their use in pharmaceutical compositions

(76) Inventors: Alessandra Nardin, Paris (FR); Andrew Kaiser, St Andre Les (FR); Claire Boccaccio, Paris (FR); Sylvie Jacod, Bry Sur Marne (FR); Jean-Pierre Abastado, Paris (FR)

Correspondence Address: YOUNG & THOMPSON, 745 SOUTH 23RD STREET, 2ND FLOOR, ARLINGTON, VA, 22202, US

(21) Appl. No.: 10/484,382

(22) PCT Filed: May 16, 2002
(86) PCT No.: PCT/EP02/05411

(30) Foreign Application Priority Data
Jul. 25, 2001 (EP) 01402010.1

(51) Int. Cl. A61K 48/00; C12N 5/08

(52) U.S. Cl. 424/93.21; 424/93.71; 435/372

(57) ABSTRACT

The invention relates to dendritic cells irreversibly triggered to maturation, which are CD14 positive, which express MHC class I with a median fluorescence intensity less than about 1500 and CD86 with a median fluorescence intensity less than about 500, as determined by immunofluorescence staining and flow cytometry analysis.

The present invention relates to new isolated dendritic cells, a process for preparing the same and their use in pharmaceutical compositions.

Dendritic cells are defined as the most potent antigen presenting cells able to stimulate both primary and secondary immune responses against specific exogenous antigen (Hart “Dendritic cells: unique leucocyte populations which control the primary immune response” Blood, 1997, vol.90,p3245). In vivo, immature dendritic cells that have captured antigens in the periphery migrate through lymphatic vessels to T cell zones of lymphoid organs where they present epitopes deriving from these antigens in the context of MHC molecules and allow activation and proliferation of antigen-specific naive T cells.

Dendritic cells can be obtained from different tissue sources or from precursors present in blood or in bone marrow. Immature dendritic cells may be obtained from blood cells by differentiating monocytes using defined culture conditions. As an example monocyte derived dendritic cells can be prepared according to patent applications WO 94/26875, WO 96/22781, or WO 97/44441. Immature dendritic cells may also be prepared according to. Boyer et al. (Exp. Hematol. 1999, vol.27, pp751-761) or to Banchereau et al (Annu. Rev. Immunol., 2000, 18:767-911).

After being differentiated from blood monocytes, for example in presence of IL-13, dendritic cells present an immature phenotype: in particular they do not secrete (or secrete very few) IL-10 and IL-12.

Immature dendritic cells do not spontaneously mature in vitro. Without maturation agents, they die or return to macrophage type cells.

In vivo, during migration, dendritic cells undergo a maturation process that results in morphological and phenotypical changes. Maturation induces a reduced capacity of dendritic cells to capture antigens and an increased capacity of antigen presentation. The production of cytokines is a property which is acquired upon dendritic cells maturation. Maturated dendritic cells are capable to secrete immunostimulatory cytokines, like the bioactive form of IL-12 (IL-12p70) and IL-10. IL-12 is able to polarize the immune system towards a T helper 1 (Th1) cytotoxic response, whereas IL-10 induces a T helper 2 (Th2) response.

A Th1 type response is considered as an immune response, involving stimulation of antigen specific T lymphocytes CD8+, whereas a Th2 type immune response involves rather a stimulation of antibody response and possibly unresponsiveness of the cytotoxic lymphocytes to an antigen.

T helper 1 CD4 T cells will help cytotoxic CD8 T cells response, T helper 2 will help B cells for antibody production. A global equilibrium exists between the two types of response. Th2 cytokines like IL-4, IL-5 or IL-10 might interfere with Th1 responses, cause energy and other various immunosuppressive effects. It is generally believed that in cancer or infectious diseases, the most effective responses are cytotoxic (CD8 T cells killing the tumor cell or the infected cell), rather than humoral responses (antibodies).

There is currently a growing interest in the use of autologous antigen, loaded dendritic cells for the treatment of cancer and infectious diseases, as it appears to be a safe, well-tolerated and potentially effective cellular therapy. It is recognized that dendritic cells, depending on their stage of maturation and on the pattern of cytokines secreted, are powerful modulators of T cell responses.

Possibilities for preparing ex vivo large quantities of dendritic cells have recently been developed, followed by a growing interest for the use of these cells in immunotherapy and as cellular vaccines.

One of the aims of the invention is to produce new dendritic cells which favor a Th1 immune response.

Another aim of the invention is to obtain new isolated dendritic cells, which can be easily and quickly obtained.

Another aim of the invention is to provide new dendritic cells which can be preserved until use and injected to a patient at appropriate timing, while preserving optimal activity.

The invention relates to dendritic cells irreversibly triggered to maturation which present the following characteristics:

they are CD14 positive,

they express MHC class I with a median fluorescence intensity of less than about 1500 and CD86 with a median fluorescence intensity of less than about 500, as determined by immunofluorescence staining and flow cytometry analysis.

The term “maturation” is defined as the activation of immature highly phagocytic dendritic cells, resulting into phenotypic and functional modification of the cells. In particular, cells acquire the property to produce high levels of cytokines and lose their phagocytic capacity.

The term “dendritic cells irreversibly triggered to maturation” is defined as cells which have immature characteristics, such as low secretion of cytokines and expression of CD14, but which will mature in any cell culture medium.

The term “median fluorescence intensity” is defined as relative fluorescence intensities for each fluorochrome in a cell sample. Monoclonal antibodies used for the detection are directly coupled to fluorochromes (Boyer et al., Exp. Hematol., 27, 751-761, 1999).

A flow cytometer is designated to detect relative differences in these parameters, and does not provide absolute data in terms of unit measurements. According to FACS™ Academy, from Becton Dickinson Immunocytometry Systems, Computer Based Training, Volume I,“Flow Cytometry and Immunology Basics” : “Flow cytometer detects differences in size, relative granularity and fluorescence, if any, associated with particles. [. . . ] A beam of laser light is projected across the cells. At the same time laser light hits the particle, any fluorescence present in or on the particle will fluoresce. Once excited, the intensity of fluorescence signal emitted should be proportional to the amount of the fluorescence compound in the particle.”

The triggered dendritic cells of the invention are CD14 positive, express MHC class I with a median fluorescence intensity less than about 1500 and CD86 with a median fluorescence intensity less than about 500, all of these properties corresponding to immature characteristics.

The dendritic cells of the invention secrete low levels of IL-12p70 and of IL-10, such as immature dendritic cells. According to a particular embodiment of the invention, dendritic cells irreversibly triggered to maturation secrete less than about 3000 pg/ml of IL-12p70 and less than 500 pg/ml of IL-10, as determined by Elisa assay for 10⁶ cells/ml.

According to a more particular embodiment of the invention, the dendritic cells irreversibly triggered to maturation present the following characteristics:

they are CD14 positive,

they express MHC class I with a median fluorescence intensity of less than about 700 and CD86 with a median fluorescence intensity less than about 300 (as determined by immunofluorescence staining and flow cytometry analysis),

and they secrete less than about 80 pg/ml of IL-12p70 and less than about 300 pg/ml of IL-10 (as determined by Ellsa assay for 10⁶ cells/ml).

According to a particular embodiment, the dendritic cells irreversibly triggered to maturation of the invention are CD83 negative. This absence of expression is a characteristic of an immature phenotype.

According to another embodiment, the dendritic cells of the invention present the following characteristics:

CD83 expression with a median fluorescence intensity from about 3 to about 20

CD14 expression with a median fluorescence intensity from about 20 to about 100

MHC Class I expression with a median fluorescence intensity from about 400 to about 700

CD86 expression with a median fluorescence intensity from about 100 to about 300 as determined by immunofluorescence staining and flow cytometry analysis, and:

IL-12p70 secretion of 1 to about 80 pg/ml

IL-10 secretion of about 15 to about 300 pg/ml. as determined by ELISA assay, for a total of 10⁶ cells/ml.

Dendritic-cells-irreversibly triggered to maturation present characteristics of both mature and immature phenotypes.

Expression range of CD83, absence of CD14 and high secretion of IL-12 are characteristics of mature phenotype.

Expression range of CD14 and absence of CD83 are characteristic of immature phenotype (For review see “Banchereau et al., 2000, Ann. Rev. Immunol.).

Dendritic cells of the invention are originated from immature dendritic cells derived from blood monocytes cultured for 1 to 16 hours, preferably 6 hours.

According to another embodiment, the dendritic cells of the invention have properties such that they can be arrested in their maturation process, and that they can resume maturation after this arrest, when cultivated in appropriate conditions.

This property is very interesting because said dendritic cells can be injected into a patient, for example for treatment of cancer. In particular, autotogous cells taken from the patient can be treated with maturation factors to obtain dendritic cells of the invention, which can be preserved until injection. They will achieve full maturation in the patient's tissues.

According to another embodiment of the invention, said dendritic cells have the properties of becoming mature in vitro in a culture medium containing no maturating factors and no cytokines, for a sufficient culture time.

The expression “containing no maturation factors and no cytokines” means that the culture medium can be devoid of the generally used maturation factors and cytokines such as IFNγ, poly I:C, CD40 ligand or antibody, lipopolysaccharide, TNFα, FLAT 3 ligand, . . . .

The expression “sufficient culture time” means a time sufficient for said cells to acquire characteristics of mature dendritic cells, in fact 16 h to 40 h. Generally speaking, it means that these cells present a reduced capacity to capture antigens and an increased capacity of antigen presentation. In particular, maturated cells have acquired capacity to secrete more than about 1000 pg/ml of IL-12p70 and to induce Th1 immune response.

According to another embodiment of the invention, said dendritic cells have the properties of becoming mature iii vivo after injection to a patient.

When injected in vivo to patients, said dendritic cells will complete their maturation and produce the cytokines required for effective specific T cells stimulation. Serum of the patient is an appropriate medium for maturation of said cells. Once injected, dendritic cells migrate to T cell areas following the progression of their activation. The present invention purposes enable the practitioner to inject optimally effective dendritic cells for the induction of cytotoxic T cell immune response.

The present invention also relates to dendritic cells, loaded with a drug, a nucleic acid or an antigen of interest, for example a tumoral antigen. The dendritic cells of the invention can also be loaded with lysates of tumor cells, in particular melanoma tumor cell lines (See example 9).

Cells may be loaded with an antigen by phagocytosis, pinocytosis, affinity binding, fusion, nucleic acid (DNA, RNA) transfer or receptor mediated uptake, according to methods known by a man skilled in the art.

According to another embodiment, the dendritic cells of the invention promote the development of T helper CD4+ T cells, and activate cytotoxic CD8+ T lymphocytes specific for an antigen, after previous contact between said antigen and phagocyting dendritic cells.

Activation of cytotoxic CD8+ T lymphocytes induces secretion of IFNγ by these cells. Thus, induction of Th1 response can be followed by Elispot assay for IFNγ, release.

Functionally, these dendritic cells are very powerful in the in vitro generation of antigen-specific CD8 T cell even in the absence of CD4 help and without additional exogenous cytokines, conditions in which immature dendritic cells are not capable of generating and sustaining a CD8 T cell response (See examples 5 and 6).

The present invention also relates to a composition of dendritic cells irreversibly triggered to maturation, under frozen form in an appropriate cryopreservative medium.

An appropriate medium is for example composed of 10% autologous serum+10% Idimethylsulfoxide in phosphate buffer saline (See example 9).

Cells of the invention can be kept frozen at temperatures below −80° C. until use, which is an industrial advantage.

The present invention also relates to a process for obtaining dendritic cells irreversibly triggered to maturation, which present the following characteristics: they are CD14 positive, they express MHC class I with a median fluorescence intensity inferior to about 1500 and CD86 with a median fluorescence intensity inferior to about 500 (as determined by immunofluorescence staining and flow cytometry analysis), comprising the step of contacting immature dendritic cells derived from blood monocytes and incubating them for 1 to 16 hours, preferably 6h, with a combination of two factors:

a—Cytokine or agonist of cytokine or cytokine inducing factor,

and b—Bacterial mixture of membrane fractions and/or ribosomal fractions, or ligand or an agonist, said ligand or its agonist being different from a cytokine.

In a particular embodiment of the invention, the process as described leads to obtain dendritic cells irreversibly triggered to maturation, which present the following characteristics they are CD14 positive, they express MEC class I with a median fluorescence intensity inferior to about 700 and CD86 with a median fluorescence intensity inferior to about 300 (as determined by immunofluorescence staining and flow cytometry analysis), and they secrete less than about 80 pg/ml of IL-12p70 and less than about 300 pg/ml of IL-10 (as determined by Elisa assay for 10⁶ cells/ml).

Combination of two factors is preferably composed of a cytokine, like IFNγ and a bacterial membrane fraction.

By means of kinetics studies, we demonstrated that a short in vitro contact of immature dendritic cells with maturation agents is sufficient to trigger a maturation process that then proceed and fully complete maturation spontaneously after removal of the maturation agents. As shown in example 6, 6 hours incubation is the optimal time to produce dendritic cells able to generate CD8 cells which are specific for given antigen. As shown in examples 4 and 8, a short time incubation with maturation agents is not sufficient to induce high levels of IL-10 secretion.

After 16 hours of incubation, obtained cells show mature characteristics and thus do not fulfill the definition of dendritic cells irreversibly triggered to maturation.

The term “bacterial membrane” is defined as the internal cytoplasmic membrane and includes the intermembranal space, and exclude the external membrane. Schematic representation of membrane is shown in “Drugs, 1997, Adis international limited”, p34 FIG. 2.

The term “membrane extracts” is defined as bacterial extracts enriched in the same membrane fractions. Membrane extracts or fractions correspond to any extract or fraction containing these membranes, purified or partially purified from a bacterial culture. The process of preparation of such extracts comprises at least a step of lysis of the bacteria obtained after the culture, and a step of separation of the fraction containing bacterial membranes, in particular by centrifugation or filtration.

The term “ribosomal extracts” is defined as bacterial extracts containing ribosomal fractions, and particularly single and/or double stranded ribonucleic acid. Ribosomal extracts or fractions correspond to any extract containing ribosomes, purified or partially purified from a bacterial culture. The process of preparation of such extracts comprises at least a step of lysis of the bacteria obtained after the culture, and a step of separation of the fraction containing bacterial ribosomes from the total lysate, in particular by centrifugation or filtration.

There are several known agents used for the maturation of dendritic cells, such as poly IC, ligands of CD40, anti-CD40 antibodies, endotoxins, living bacteria, lipopolysaccharide, culture supernatants and cocktail of agonistic cytokines, including TNFα. The interferon-γ acts in synergy with the maturation agent to increase maturation characteristics of dendritic cells and their stimulating phenotype.

Ribomunyl® (International Non-proprietory Name, or Generic name: Ribosomal and membranar bacterial fractions, membranar proteoglycanes) is known for its non specific natural immnunostimulatory effect. It contains both proteoglycans from Klebsiella pneumoniae (0,015 mg in a dose of lyophylisate) and ribosomal fractions containing 70% RNA from 4 different bacterial strains, Klebsiella pneumoniae (35 parts), Streptococcus pneumoniae (30 parts), Streptococcus pyogenes group A (30 parts) and Haemophilus influenzae (5 parts) (0,01 mg of ribosomal extracts in a dose of lyophylisate). Ribomunyl was shown to stimulate the general innate immune response by acting on polymorphonuclear cells (PMNs) and macrophages, to increase the production of several cytolines (IL-1, IL-6, IL-8, TNFα, CSF), and to be able to activate natural killer cells.

Heat shock protein 70 has been identified as a potential maturation factor for some monocytes (Kuppner et al., 2001); it can effectively be combined to poly I:C or to a cytokine to trigger immature dendritic cells to maturation.

The present invention also relates to a process for the preparation of dendritic cells irreversibly triggered to maturation, which are CD83 negative.

In a particular embodiment of the invention, the used cytokine in the process may be IFNγ and the used cytokine inducing factor may be poly I:C.

Poly I:C induces secretion of IFNγ.

In a particular embodiment of the invention, the used bacterial mixture of membrane fractions and/or ribosomal fractions is a membrane subfraction of one strain of bacteria. In another embodiment, the membrane subfraction can be a purified protein obtained from said membrane subfraction.

In a particular embodiment of the invention, the used bacterial mixture of membrane and/or ribosomal fractions is Ribomunyl® (Inava Laboratory, Pierre Fabre).

Combination of Ribomunyl and IFNγ is more efficient to induce IL-12 secretion by dendritic cells than use of Ribomunyl alone (see example 3).

In a particular embodiment of the invention, the used membrane subfraction is FMKp (Klebsiella pneumoniae membrane fraction). The use of FMKp until Ribomunyl in the process of the invention leads to a more important secretion of cytokines by obtained dendritic cells (Compare results of examples 1C, 3A and 3B with example 7C).

In particular the used cytokine in the process is IFNγ and the membrane subfraction is FMKp. Preferably the concentration of IFNγ is about 500 U/ml and the used concentration of FMKp is about 1 μg/ml.

In a particular embodiment of the invention, the used ligand is an antibody anti-CD40 or a CD40 ligand.

In a particular embodiment of the invention, the used ligand is an inducible Heat shock protein 70 or isolated polypeptide sequences from it.

The present invention also concerns the irreversibly triggered dendritic cells -liable to be obtained according to the process described in the present application. The invention also relates to mature dendritic cells which have a secretion of IL-12p70 higher than secretion of IL-10, i.e. where ratio IL-12p70 /IL-10 secretion is superior than 1. In particular the invention relates mature dendritic cells which secrete more than about 1000 pg/ml of IL-12p70 and less than about 100 pg/ml of IL-10 (as determined by Elisa assay for 10⁶ cells/ml) for at least 24 hours, and stimulate Th1 and cytotoxic immune response.

The associated phenotypic modifications are the increase in CD80, CD86, CD83, MHC class I and II molecules cell surface expression and the decrease in CD14 surface expression. The functional changes are the loss of phagocytic properties, the acquisition of migration abilities, and changes in the cytokine and chemokine expression profile, and particularly an increased IL-12 secretion.

The invention also relates to a process for preparing mature dendritic cells from dendritic cells irreversibly triggered to maturation, which comprises a step of culture of said irreversibly triggered dendritic cells, without exogenous maturation factor nor cytokine added, in vitro or in vivo.

The invention also relates to the above defined mature dendritic cells such as obtained by the process.

The present invention also relates to pharmaceutical compositions containing as active substance dendritic cells irreversibly triggered to, maturation, having interiorized antigens, preferably vaccinal antigens, -in association with a pharmaceutically acceptable vehicle.

The term “vaccinal antigens” is defined as antigens inducing an immune response.

The dendritic cells according to the invention are able to act on precise T cells subpopulations. This means that the dendritic cells according to the invention are able to stimulate or to regulate Th2/Th1 immune response. Dendritic cells described in the state of the art are able not only to induce an in vivo antigen-specific proliferation of T cells, thus leading to an antigen specific increased cytotoxicity and imnmunostimulation, but also to induce in vivo regulatory T cells and therefore inhibition of antigen-specific cytotoxic T cells, leading to unresponsiveness to a specific antigen

Dendritic cells of the invention possess only strong immunostimulatory properties via Th1 immune response mainly and are suitable for clinical vaccinal use.

An induced immune response might be characterized by an in vivo clinical immune response against a given pathogen or a tumour, leading to its decrease or its elimination. In vitro, this may be measured, for dendritic cells, in a immunostimulation assay of antigen-specific CD8 cytotoxic T lymphocytes.

The present invention also relates to cellular vaccine composition containing as active substance dendritic cells irreversibly triggered to maturation, in a amount of about 10⁴ to about 10⁹, and preferably about 10⁵ to about 10⁷ of said cells per vaccinal dose.

The term “cellular vaccine composition” is defined as dendritic cells having processed and presenting vaccinal antigens.

### LEGENDS OF THE FIGURES

Abbreviations used:

Anti-CD40 mAb=monoclonal Antibody against CD40

DC=Dendritic cell

FMKp=Klebsiella pneunomiae membrane fraction

HLA-ABC=Histocompatibility Class I Molecules

iDC=Immature dendritic cell

IFNγ=Interferon gamma

Isot Ctr=Isotype control

IVS=in vitro stimulation

MFI=Median Fluorescence Intensity

Poly I:C=Polyriboinosinic Polyribocytidylic Acid

RBL=Ribomuny^{R}

FIGS. 1A, 1B, 1C: Ribomunyl^{R} and IFNγ triggered dendritic cells are committed to become fully mature

Immature dendritic cells were incubated for 6 h with Ribomunyl (RBL)+IFNγ, washed, and further cultured for 34 h.

White bars represent measures at time point 6 h (after 6 h triggering with RBL+IFNγ), black bars represent measures at time point 40 h (after 6 h triggering with RBL+IFNγ and 34 h of culture without any maturation factor).

To follow maturation, dendritic cells were stained with anti-CD14, anti-CD83, anti-HLA ABC and anti-CD86 antibodies, and the fluorescence analysed with flow cytometer. The expressions of the markers CD14, CD83 (1A), HLA-ABC and CD86 (1B) at time points 6 h and 40 h are expressed as Median Fluorescence Intensity arbitrary units (Y axis).

Concentration of IL-10 and IL-12p70 in culture supernatants was measured at time points 6 h and 40 h by ELISA (1C), and is expressed in pg/ml/10⁶ cells (Y axis).

FIGS. 2A, 2B, 2C: Poly I:C and antibody anti-CD40 triggered dendritic cells are committed to become fully mature

Immature dendritic cells were incubated for 6 h with anti-CD40 mAb+Poly I:C, washed, and firer cultured for 34 h.

White bars represent measures at time point 6 h (after 6 h triggering with poly I:C+anti-CD40 mAb), black bars at time point 40 h (after 6 h triggering with poly I:C+anti-CD40 mAb and 34 h of culture without any maturation factor).

To follow maturation, dendritic cells were stained with anti-CD14, anti-CD83, anti-HLA-ABC and anti-CD86 antibodies, and the fluorescence analysed with flow cytometer. The expression of the markers CD14, CD83 (2A), HLA-ABC and CD86 (2B) is expressed as Median Fluorescence Intensity arbitrary units (Y axis).

Concentration of IL-10 and EL-12p70 in culture supernatants was measured at time points 6 h and 40 h by ELISA (2C), and is expressed in pg/ml/10⁶ cells (Y axis).

FIGS. 3A, 3B : Comparison of maturation effects of Ribomunyl alone and Ribomunyl in association with IFNγ on cytokines secretion by dendritic cells

Immature dendritic cells were incubated 6 h with RBL alone or RBL+IFNγ, washed, and further cultured for 34 h.

White bars represent measures at time point 6 h (after 6 h triggering with RBL with or without IFNγ), black bars at time point 40 h (after 6 h triggering: with RBL with or without IFNγ, and 34 h of culture without any maturation factor).

Quantities of cytokines IL-12p70 (3A) and IL-10 (3B) in culture supernatants were measured at time points 6 h and 40 h, by ELISA. Concentration of cytokines is expressed in pg/ml/10⁶ cells (Y axis),

FIG. 4: IL-10 secretion requires longer incubation times of dendritic cells in presence of RBL+IFNγ

Immature dendritic cells were incubated:

6 h with RBL+IFNγ, washed, and further cultured 34 h,

or 40 h with RBL+IFNγ.

Quantity of secreted IL-10 in culture supernatants was measured by ELISA:

After 6 h of stimulation with RBL+IFNγ (black bar/white points)

After 40 h of culture, comprising 6 h of stimulation with RBL+IFNγ (white bar/black points)

After 40 h of stimulation with RBL+IFNγ (white bar/black shading)

Concentration of IL-10 is expressed in pg/ml/10⁶ cells (Y axis).

FIG. 5: Triggered dendritic cells pulsed with peptide antigen can generate antigen specific CD8 T cells even in absence of exogenous cytokines.

Immature dendritic cells were incubated for 6 hours in absence of maturation stimuli (iDC), in presence of anti-CD40 mAb+Poly I:C or of RBL+IFNγ. Cells were pulsed with peptide just before the end of maturation time, then harvested, washed and used to stimulate CD8 T cells. After in vitro stimulation with peptide-pulsed dendritic cells, IFNγ release by CD8 T cells was assayed by ELISPOT assay using T2 cells pulsed with Melan A peptide as stimulators.

Different symbols represent each donor. Bars show average values for each condition of stimulation. Y axis shows number of specific spot forming cells.

FIG. 6: Dendritic cells triggered by a 6 h incubation with RBL+IFNγ are optimal for the generation of antigen specific effector CD8 T cells

Immature dendritic cells were incubated in absence of maturation stimuli (iDC) or in presence of RBL+IFNγ for 3, 6 or 16 hours. Cells were pulsed with peptide just before the end of maturation time, then harvested, washed and used to stimulate CD8 T cells. After 2 stimulations with peptide-pulsed dendritic cells, IFNγ release by CD8 T cells was assayed by ELISPOT assay using T2 cells pulsed with Melan A peptide as stimulators.

Different symbols represent each donor. Bars show average values for each condition. Y axis shows number of specific spot forming cells.

FIGS. 7A, 7B, 7C: FMKp and INFγ triggered dendritic cells are committed to become fully mature

Immature dendritic cells were treated with FMKp and IFNγ for 6 hours. Cells were washed at 6 hours and analysed immediately or further cultured in absence of maturation factors until the 24 hours time point.

White bars represent measures at time point 6 h (after 6 h triggering with FMKp+IFNγ), black bars represent measures at time point 24 h (after 6 h triggering with FMKp+IFNγ and 18 h of culture without any maturation factor.

To follow maturation, dendritic cells were analysed for their expression of markers CD14 and CD83 (1A), and HLA-ABC and CD86 (1B) at both time points. Expression of the markers is expressed as median fluorescence intensity arbitrary units (Y axis).

Concentration of IL-12p70 and IL-10 in culture supernatants was measured at both time points by ELISA (1C) and is expressed in pg/ml/10⁶ cells (Y axis).

FIG. 8: IL-10 secretion by dendritic cells requires a long time incubation of cells in presence of FMKp+IFNγ

Immature dendritic cells were treated with FMKp and IFN-γ for 3 h, 6 h or 40 h. Cells stimulated 3 h or 6 h were washed and either analysed at time point 3 or 6 hours, or further cultivated in medium without any maturation factor, and IL-10 secretion was then measured at time point 24 hours.

Quantity of secreted IL-10 in culture supernatants was measured by ELISA:

after 3 h of stimulation with FMKp+IFNγ (white bar)

after 6 h of stimulation with FMKp+IFNγ (white bar/black points)

after 24 h of culture comprising 3 h of stimulation with FMKp+IFNγ (black bar)

after 24 h of culture comprising 6 h of stimulation with FMKp+IFNγ (black bar/white points)

after 40 h of stimulation with FMKp+IFNγ (white bar/black shading).

Concentration of IL-10 is expressed in pg/ml/10⁶ cells (Y axis).

FIG. 9: Loading of dendritic cells with melanoma cell lysate followed by freezing/thawing steps do not perturb the maturation process

Immature dendritic cells were loaded with Colo829 lysate by overnight incubation, and then treated with FMKp and IFN-γ for 6 hours. After washes, cells were frozen in 4% Albumin/DMSO and then thawed.

Freshly thawed cells were resuspended in albumin 4%, incubated for 2 h at 4° C. in 1 ml 20 syringes, passed through a 25 G needle after shaking of the syringes, and cultured 18 hours in complete AIMV medium without any maturation stimuli.

IL-12p70 and IL-10 secretions were assayed by ELISA:

after loading with Colo829 lysate, and 6 h incubation with FMKp and IFNγ, (white bars)

after loading with Colo829 lysate, 6 h incubation with FMKp and IFNγ, freezing, thawing, and 18 h culture in AIMV medium (black bars).

Concentration of IL-12p70 and IL-10 in culture supernatants is expressed in pg/ml/10⁶ cells (Y axis).

### EXAMPLES

Abbreviations Used:

Anti-CD40 mAb=monoclonal Antibody against CD40

DC=Dendritic cell

FMKp=Klebsiella pneunomiae membrane fraction

HLA-ABC=Histocompatibility Class I Molecules

IDC=Immature dendritic cell

IFNγ=Interferon gamma

IVS=in vitro stimulation

MFI=Median Fluorescence Intensity

PBS=Phosphate buffer saline

Poly I:C=Polyriboinosinic Polyribocytidylic Acid

RBL=Ribomunyl^{R}

### Example 1

### Ribomunyl^{R} and IFNγ Triggered Dendritic Cells are Committed to Become Fully Mature after ex vivo Culture

Dendritic Cells:

Immature dendritic cells were prepared by culture of peripheral blood monocytes and elutriated, according to the patent applications WO 97/44441, and to Boyer et al. (Exp. Hematolo., 1999, 27, 751-761). Briefly, dendritic cells were differentiated in AIMV medium supplemented with 500 U/ml GM-CSF (Leucomax, Novartis Pharma) and 50 ng/ml IL-13 (Sanofi Synthelabo) (=complete AIMV medium), and elutriated after 7 days of culture.

Maturation:

Dendritic cells were cultured in complete AIMV medium (Life Technologies, Paisley PA49RF, GB) for 6 hours in presence of Ribomunyl (1 ug/ml) and IFNγ (500 U/ml). Each vial of lyophilised RiboMunyl^{R} (Inava Laboratory, Pierre Fabre, Paris, France) contains 0.010 mg of ribosomal fractions from K. pneumoniae, S. pneumnoniae, S. pyogenes and H. influenzae, and 0.015 mg of membrane fractions from K. pneumoniae. IFNγ (Imukin) was obtained from Boehringer Ingelheim France. Cells were washed at 6 hours and further cultured in complete AIMV medium (in absence of maturation factors) until the 40 hours time point.

Phenotypic Analysis:

To follow maturation, dendritic cells were analysed for their expression of CD83, CD86, CD14 and HLA-ABC markers at time points 6 h and 40 h. Dendritic cells were suspended in phosphate buffer saline (PBS) supplemented with 1% foetal calf serum. Cells were incubated for 30 mn on ice with the following FITC or PE-conjugated specific monoclonal antibodies or isotype matched controls : anti-CD83, CD86, MHC Class I, mouse IgG2b, mouse IgG2a (Immunotech, Marseille, France), anti-CD14 (Becton Dickinson, St Jose, Calif.). Cells were then washed in PBS and resuspended in PBS containing TO-PRO 3 at 3 nM, to exclude death cells from analysis.

Flow cytometry analysis was performed with a Becton Dickinson cytometer with a CellQuest software. Results are expressed as Median Fluorescence Intensity (MFI) values (arbitrary units).

Cytokine Detection:

At time points 6 h and 40 h, culture supernatants were assayed by ELISA for IL-12p70 and IL-10 secretion by commercial ELISA, performed using antibody pairs from R&D Systems Europe (Abingdon, UK) according to manufacturer's instructions.

Results:

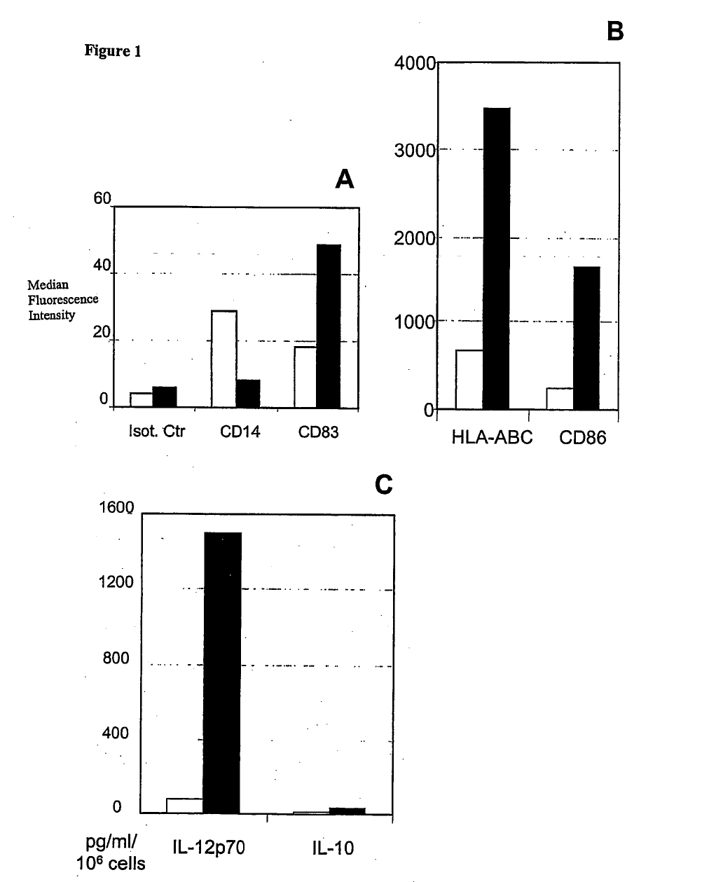

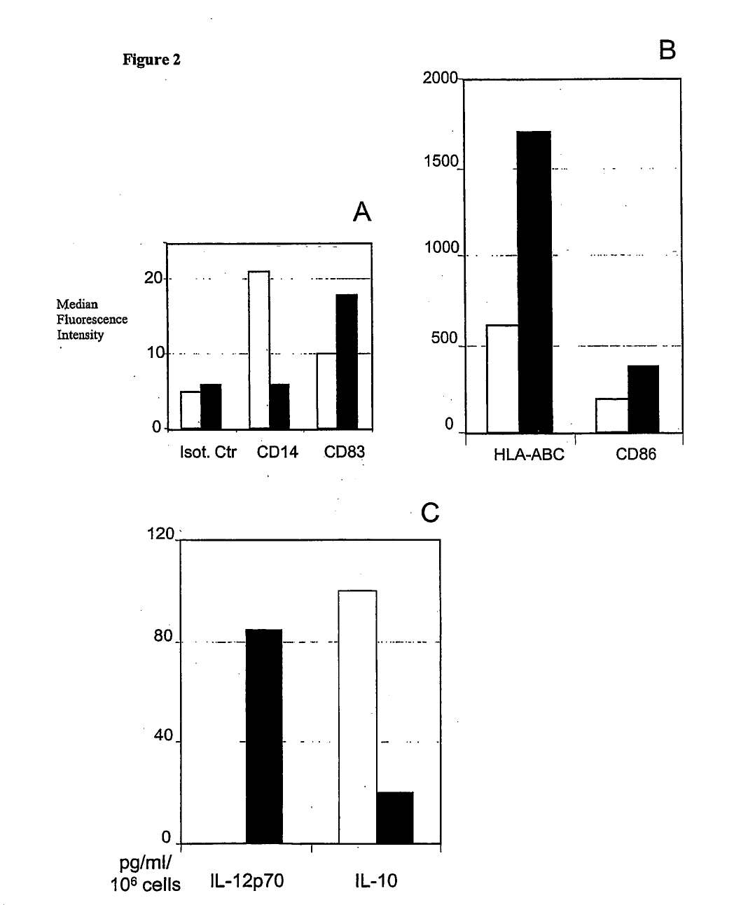

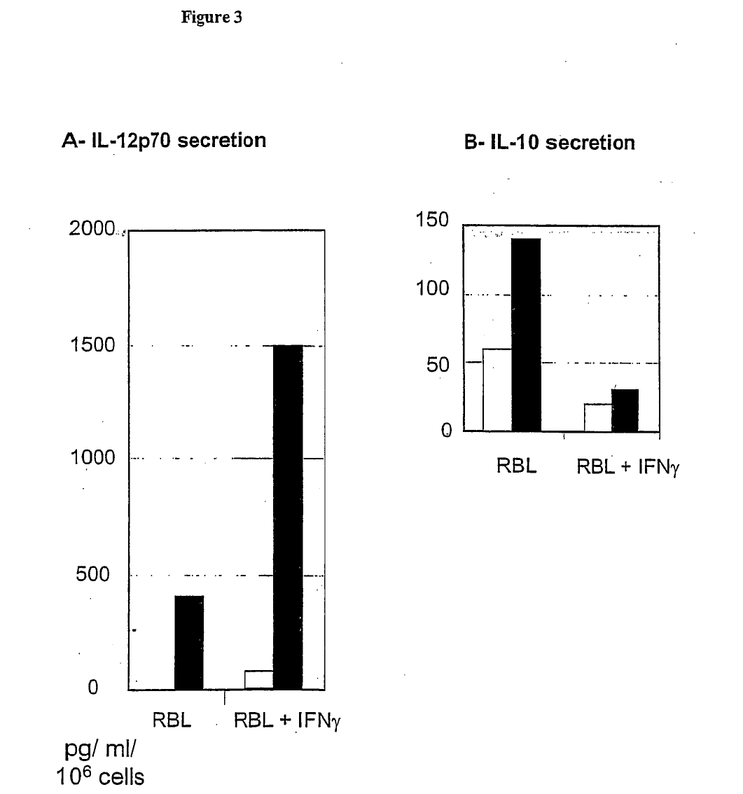

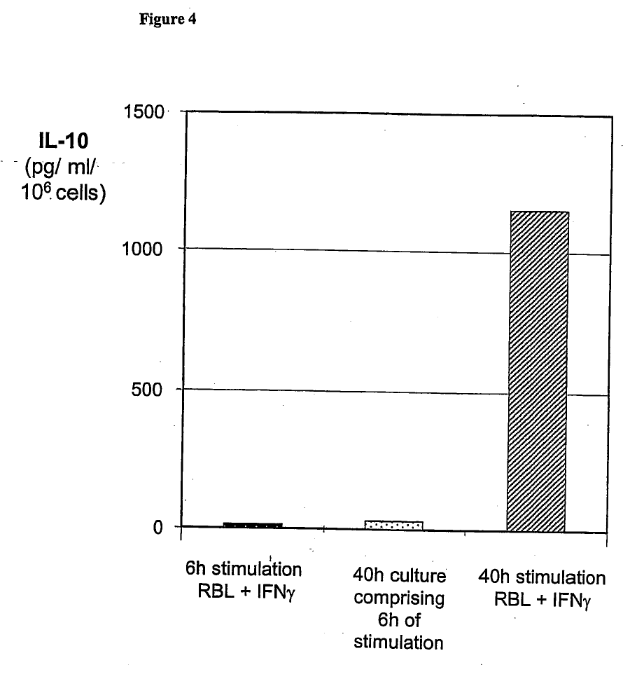

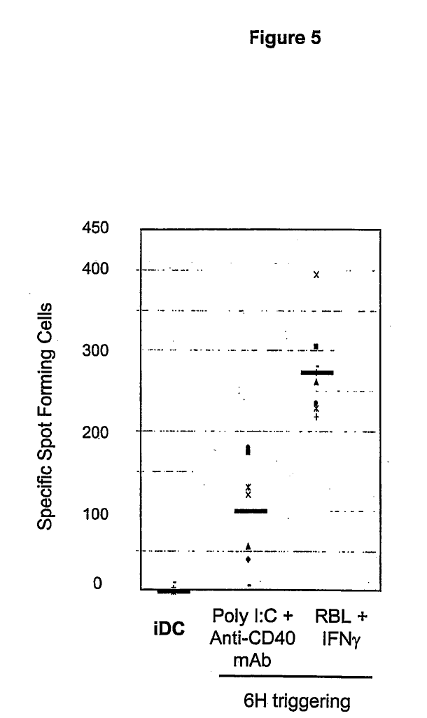

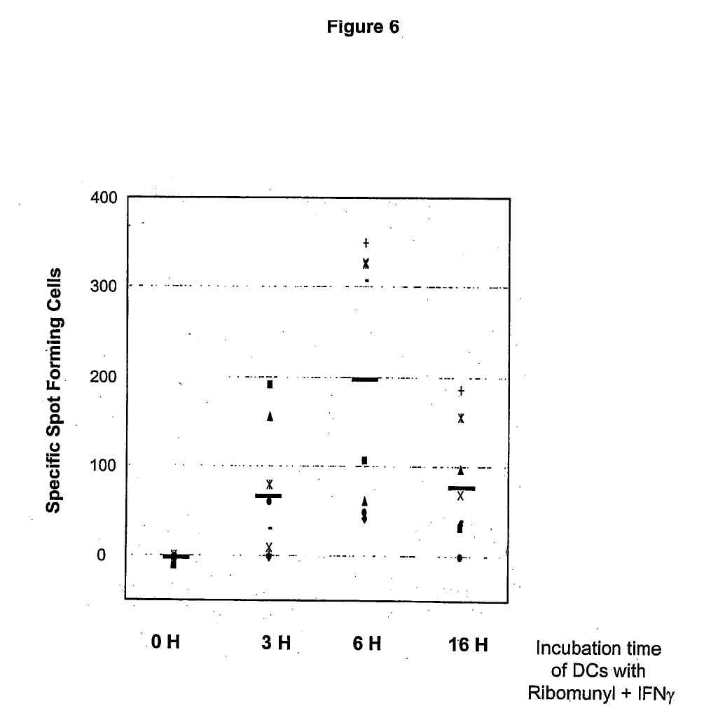

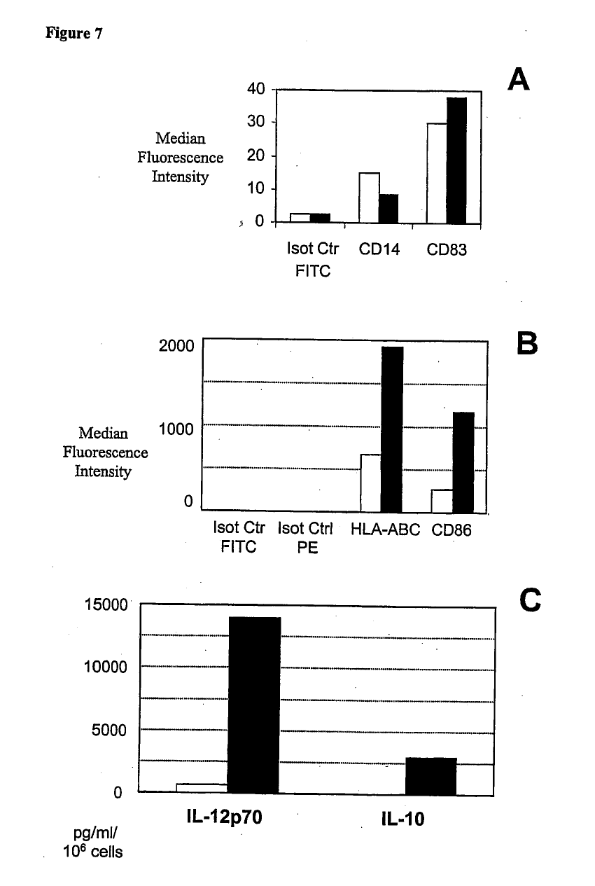

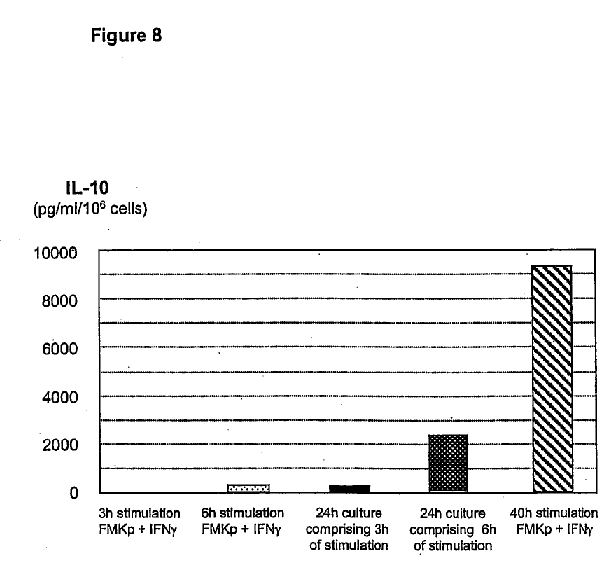

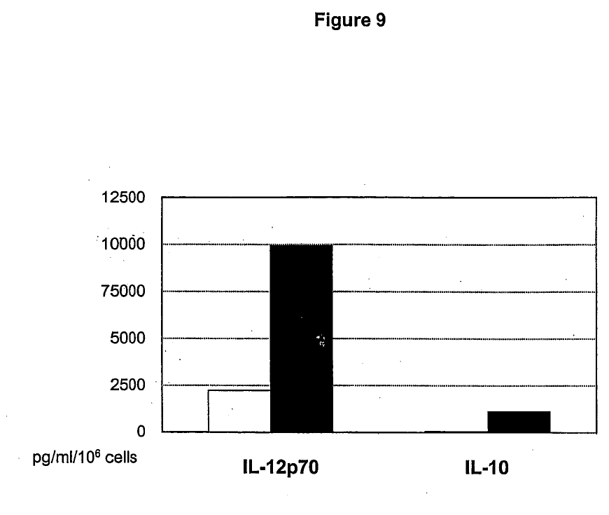

Results are presented on FIGS. 1A—CD14 and CD83 expression; 1B—HLA-ABC and CD86 expression; 1C—cytolines secretion. Negative controls (isotype controls) for FACS analysis are shown.

After 6 h of incubation with maturation factors, dendritic cells present immature cells characteristics: cells express CD14 but very low quantities of CD83. HLA-ABC and CD86 are not significantly upregulated. Secretion of IL-12p70 and IL-10 is very low.

After wash and further culture until 40 h, cells present mature cells characteristics: they are CD14 negative, express CD83 with a fluorescence intensity of 49 (about 95% positive cells), HLA-ABC with fluorescence intensity of 3460 and CD86 with fluorescence intensity of 1640. IL-12p70 secretion increases dramatically, while IL-10 secretion remains low.

In conclusion, dendritic cells become mature during culture in absence of maturation factors, after they have been. triggered to maturation during a 6 h incubation with RBL+IFNγ.

### Example 2

### Poly I:C and Antibody anti-CD40 Triggered Dendritic Cells are Committed to Become Fully Mature after ex vivo Culture

Immature dendritic cells were prepared as described in example 1.

Dendritic cells were incubated in complete AIMV medium for 6 hours in the presence of antibody anti-CD40 (2 μg/ml) and poly I:C (100 μg/ml), then washed and further cultured for 34 h.

Phenotype and cytokines secretion were analysed as described in example 1.

Results:

Results are presented on FIGS. 2A—CD14 and CD83 expression; 2B—HLA-ABC and CD86 expression; 2C—cytokines secretion. Negative controls (isotype controls) for FACS analysis are shown.

As demonstrated for combination RBL+IFNγ, a 6 h incubation with anti-CD40 mAb and poly I:C was sufficient to trigger dendritic cells maturation. At the 6 h time point, cells present immature characteristics although after wash and 34 h culture with no maturation factors, dendritic cells express high levels of HLA-ABC, secrete IL-12p70 (85 pg/ml) but low quantity of IL-10 (20 pg/ml).

### Example 3

### Comparison of Maturation Effects of Ribomunyl Alone and Ribomunyl in Association with IFNγ Cytokines Secretion by Dendritic Cells

Immature dendritic cells were treated for 6 h with RBL+IFNγ (as described in example 1) or with RBL alone (1 μg/ml). Cytokines secretion was assayed at the 6 h and 40 h time points, as previously described.

Results:

Results are presented on FIGS. 3A—IL-12p70 secretion; 3B—IL-10 secretion.

From 0 to 6 h (white bar), no important IL-12 secretion was induced. IL-10 secretion remains low (immature DC usually secrete low amounts of IL-10). During further 34 h of culture with no maturation factors and no cytokine, immature dendritic cells incubated with RBL alone secrete IL-12p70 (400 pg/ml/10⁶ cells) and some IL-10 (140 pg/ml), combination of RBL with IFNγ used for triggering dendritic cells to maturation leads to a better secretion of IL-12p70 (1500 pg/ml/10⁶ cells) and a reduced [IL-10 secretion (31 pg/ml). Combination of two factors permits to obtain dendritic cells which present a high ratio between IL-12p70 secretion and IL-10 secretion.

### Example 4

### Secretion of IL-10 Requires Longer Incubation Times of Dendritic Cells in Presence of RBL+IFNγ

Immature dendritic cells were treated for 6 h with RBL+IFNγ, then washed and further cultured for 34 h with no maturation factor and no cytokine, or treated for 40 h with same maturation factors. Cytokine secretion was assayed at 6 h and 40 h by ELISA.

Results:

Results are presented on FIG. 4; after 6 h treatment only 20 pg/ml of IL-10 were secreted, and only 31 pg/ml during the 34 h after wash, while 1150 pg/ml could be secreted if cells were incubated in presence of the maturation stimuli for the whole 40 h period. Thus, we conclude that long incubation times in presence of maturation stimuli will result in higher IL-10 secretion. Short time of incubation of dendritic cells leads to limited IL-10 secretion during maturation phenomen. This process of maturation permits to obtain maturated dendritic cells, which secrete mainly IL-12.

### Example 5

### Triggered Dendritic Cells Pulsed with Peptide Antigen Stimulate Antigen Specific T Cells to Release IFNγ as Measured by ELISPOT

Immature dendritic cells were prepared as described in example 1.

Dendritic cells were cultivated or not in complete AIMV medium for 6 hours in presence of RBL+IFNγ or anti-CD40 mAb+Poly I:C. Cells were pulsed with peptide before the end of maturation time, then harvested, washed and used to stimulate CD8 T cells.

Generation of Melan-A Specific CTL

Dendritic cells were pulsed for 2 h with Melan-A peptide (10 82 g/ml) and β2 microglobulin (5 μg/ml) at 37° C., treated with mitomycin C (25 μg/ml) for the last 30 min of pulsing, and washed three times. Purified autologous CD8 T cells (1.5×10⁵/well) were mixed with peptide-pulsed DC (3×10⁴/well) in microwells in complete medium (Iscove's medium supplemented with 10% autologous serunim arginne, asparagme and glutamine) in absence of exogenous cytokines. In all experiments, 8 CD8 microcultures were stimulated for each dendritic cells condition.

On day 7, autologous dendritic cells that were thawed the day before and maturated for 6 h were pulsed with the Melan-A peptide and used to restimulate the CD8 T cells still in absence of exogenous cytokines.

ELISPOT Assay for IFNγ Release

CD8 T cells generated by 2 in vitro stimulations (IVS) with DC-peptide were added to nitrocellulose 96-well plates precoated with the primary anti-IFNγ mAb (300 CD8/well). Individual microcultures were tested in duplicate. HLA-A2 pos. stimulators (T2 or autologous EBV-B dells) pulsed with the Melan-A peptide (or PSA1 as control peptide) were added at 5×10⁴/well. T cells stimulated with, PHA-L were used as positive control. After 20 h incubation at 37° C., plates were washed, incubated with biotinylated second mAb to IFNγ and stained with Vectastain Elite kit. Spots Forming Cells were counted with ELISPOT reader. Background from T cells stimulated with target cells+control peptide was subtracted for analysis and was never important.

Results:

Results are shown in FIG. 5 and are expressed by “specific spot forming cells” for 300 CD8 T cells. In absence of exogenous cytokines, immature dendritic cells are not able to generate antigen specific effector T cells. However, dendritic cells triggered to maturation are able to prime antigen-specific CD8 T cells. Poly I:C+Ab anti-CD40 dendritic cells triggered induced a reasonable response although RBL+IFNγ triggered dendritic cells are very efficient

### Example 6

### Dendritic Cells Triggered by 6 h Incubation with RBL+IFNγ Optimally Stimulate Antigen Specific T Cells as Measured by IFNγ ELISPOT

Immature dendritic cells were treated for different times (0, 3, 6 and 16 h) with RBL+IFNγ and pulsed with Melan-A peptide as described in example 5. Briefly, cells were pulsed with peptide before the end of maturation time, then harvested, washed and used to stimulate CD8 T cells in absence of exogenous cytokines. INFγ release by CD8 T cells after 2 IVS was assayed by ELISPOT as described in example 5.

Results:

In this experiment, different times of incubation with maturation stimuli were compared, in the aim to determine the optimal triggering time. Results are shown in FIG. 6. Dendritic cells triggered by a 6 h incubation in presence of RBL+IFNγ are the most effective in the generation of antigen-specific CD8 T cells. However, DC triggered for 3 or 16hare also able to induce a CD8 T cell response.

### Example 7

### FMKp and INFγ Triggered Dendritic Cells are Committed to Become Fully Mature after ex vivo Culture

Immature dendritic cells were prepared as described in example 1.

Maturation:

Immature dendritic cells were incubated at 2×10⁶ cells/ml/well in complete AIMV medium (i.e. AIMV supplemented with GM-CSF and IL-13). They were treated with FMKp (1 μg/ml, finished by Pierre Fabre Medicament, France) and 500 U/ml of IFN-γ for 6 hours. Cells were washed at 6 hours and analysed immediately or further cultured in complete AIMV medium (in absence of maturation factors) until the 24 hours time point.

Phenotypic Analysis:

To follow maturation, dendritic cells were analysed for their expression of CD83, CD86, CD14 and HLA-ABC markers at time points 6 h and 24 h.

Cells were resuspended in PBS containing 1% foetal calf serum and 0.1% sodium azide. They were stained with the following monoclonal antibodies: HLA-ABC-FITC, CD86-PE, CD14-FITC and CD83-FITC (Immunotech) for 20 mn at 4° C. After washing in PBS, cells were resuspended in PBS containing 3 nM TO-PRO-3 (Molecular Probes) to exclude dead cells. Acquisition was done on a FACSCalibur flow cytometer (Becton Dickinson). Results are expressed as Median Fluorescence Intensity (MFI) values (arbitrary units). Negative controls for FACS analysis (control isotypes PE and FITC) are shown: Isot Ctr PE and FITC.

Cytokine Detection:

At time points 6 h and 24 h, culture supernatants were collected and stored at −80° C. After thawing they were assayed by ELISA for IL-12p70 and IL-10 secretion by commercial ELISA, performed using antibody pairs from R&D Systems Europe (Abingdon, UK) according to manufacturer's instructions. Lower limits of detection were 16 pg/ml for IL-12p70, and 31 pg/ml for IL-10.

Results:

Results are presented in FIGS. 7A—CD14 and CD83 expression;

7B—HLA-ABC and CD86 expression;

7C—Cytokines secretion.

After 6 h of incubation with FMKp and IFNγ (white bars), dendritic cells present immature cells characteristics:

Cells are CD14 positive, and in this example they are CD83 positives; in some cases they can be CD83 negatives.

Expression of HLA-ABC is lower than 1500 and expression of CD86 is lower than 500 (arbitrary values).

The secretion of IL-12p70 is low, 635 pg/ml/10⁶ cells. At this time point no IL-10 is detected.

After wash and further culture until 24 h (black bars), cells present mature characteristics:

CD14 expression is nearly suppressed, CD83 expression has been slightly upregulated.

The expression of HLA-ABC has been multiplied by-about three times (673 before the culture to 1928 after the culture) and the expression of CD86 is more than four times higher after the 18 hours culture (267 before to 1165 after).

IL-12p70 secretion has increased dramatically, about twenty times more than before the culture (635 at time point 6 h to 13953 pg/ml/10⁶ cells at time point 24 h), while IL-10 secretion remains lower than 3000 pg/ml.

In conclusion, dendritic cells irreversibly triggered to maturation acquire mature characteristics during the 21 hours culture period, even in absence of maturation factors, after they have been incubated for 6 h with FMKp+IFNγ and washed.

### Example 8

### IL-10 Secretion by Dendritic Cells Requires a Long Time Incubation of Cells in Presence of FMKp+IFNγ

Immature dendritic cells were treated with FMKp (1 μg/ml) and IFN-γ (500 U/ml) for 3 h, 6 h, or 40 h. Cells stimulated 3 h or 6 h were washed and either analysed at time point 3 or 6 hours, or further cultivated in medium without any maturation factor, and IL-10 secretion was then measured at time point 24 hours. IL-10 secretion was assayed by ELISA as previously described.

Results are Presented on FIG. 8.

After 3 hours of treatment, no EL-10 was detected. After 6 hours, 271 pg/ml/10⁶ cells of IL-10 were detected in the supernatant. After further culture in medium without any maturation factor or cytokine until time point 24 h, IL-10 secretion remains low, in any case lower than 4000 pg/ml.

On contrary incubation for 40 h with FMKp and INFγ leads to a dramatic induction of IL-10 secretion: about 9000 pg/ml after 40 hours of stimulation are measured in culture supernatants.

As previously demonstrated in example 4 with the association Ribomunyl^{R}+IFNγ, short times of incubation in the presence of FMKp and IFNγ lead to limited IL-10 secretion during the maturation process.

### Example 9

### Loading of Dendritic Cells with Melanoma Cell Lysate Followed by Freezing/Thawing Steps do not Perturb the Maturation Process

Production of Melanoma Cell Lysate:

Melanoma cells from the COLO829 cell line (ATCC number CRL-1974) were harvested, washed 2 times in sterile PBS and resuspended in sterile PBS at a concentration of 5×10⁷ cells/ml. Four cycles of freezing and thawing (F/T) were applied and the lysate was sonicated in a Misonix cup horn sonicator® (4 pulses of 2 mm followed by 1 cycle of 5 mn at maximal power). The lysate was then centrifuged in a microfuge tube 5 mn at 5000 rpm and the supernatant was aliquoted and stored at −80° C.

Cell Loading:

Immature dendritic cells were diluted in complete AIMV medium at a concentration of 2×10⁶ cells/ml and tumor lysate was added at a ratio of 0.5 tumor cell equivalent/DC. Cells were then incubated overnight in EVA bags (Stedim) at 37° C.

Maturation was performed as described in example 7, with FMKp (1 μg/ml) and IFN-γ (500 U/ml) for 6 h.

Freezing and Thawing:

Loaded dendritic cells irreversibly triggered to maturation were washed in elutriation solution (PBS-Glucose, B. Braun Medical S.A.). in bags (centrifugation at 1600 rpm, 25 mn at 4° C. without brake) and the pellet was resuspended and centrifuged in tubes (1400 rpm, 10 mn at 4° C). They were frozen in 4% albumin (90%)/DMSO (10%) at a concentration of 5×10⁶ cells/ml in cryobags.

Cells were then thawed in elutriation solution supplemented with 0.93% Albumin and 3.5% ACD-A (B. Braun Medical S.A.) to avoid cell aggregates, and centrifuged at 1600 rpm, 25 mn at 4° C. without brake.

Incubation in Syringe and Final Culture:

Freshly thawed cells were centrifuged in tubes and resuspended in Albumin 4% at a concentration of 20×10⁶ cells/ml, and incubated for 2 h at 4° C. in 1 ml syringes.

Cells were then passed through a 25 G needle after shaking of the syringes (because cells formed sediments), and cultured overnight in complete AIMV medium at a concentration of 2×10⁶ cells/ml in 24-well plates.

Cytokine secretions were assayed by ELISA as previously described, just after 6 h incubation with FMKp and IFNγ, and after the final overnight culture in AIMV medium.

Results:

Results are presented in FIG. 9.

After 6 h incubation with maturation stimuli (white bars), loaded dendritic cells secrete 2222 pg/ml/10⁶ cells of IL-12p70 and 59 pg/ml/10⁶ cells of IL-10. IL-12 secretion has already started although IL-10 secretion is not yet induced.

After a freezing/thawing step, cells are cultured without any maturation stimuli for one night, making the dendritic cells which have bet irreversibly triggered to maturation finalize their maturation process (black bars). As expected, secretion of IL-12p70 becomes 4 times more important than the IL-12p70 secretion measured before 18 hours of culture (about 10 000 pg/ml/10⁶ cells after the final culture), although IL-10 secretion remains low, in fact lower than 1200 pg/ml.

We conclude that induction of high secretion of IL-12p70 by 6 h incubation of cells with FMKp and IFNγ is effective, even if cells have been previously loaded with antigens, and even if said cells have been frozen after the 6 h incubation with maturation stimuli. Dendritic cells irreversibly triggered to maturation have the property to resume maturation after an arrest in the maturation process, when cultivated in appropriate conditions.

### References

Banchereau et al., <<Immunobiology of dendritic cells>>, 2000, Ann. Rev. Immunol., 18:767-811)

Boyer et al., <<Generation of phagocytic MAK and MAC-DC for therapeutic use: Characterization and in vitro functional properties”, 1999, Exp. Hematology, vol 27, p 751-761.

Hart et al., “Dendritic cells: unique leucocyte populations which control the primary immune response”, 1997, Blood, vol 90, p 3245).

Kuppner et al., “The role; of Hsp70 in dendritic cell maturation; Hsp70 induces the maturation of immature dendritic cells but reduces DC differentiation from monocyte precursors”, 2001, Eur. J. Immunol., 31:1602-1609.

WO 94/26875: NEW MACROPHAGES, PROCESS FOR PREPARING THE SAME AND THEIR USE AS ACTIVE SUBSTANCES OF PHARMACEUTICAL COMPOSITIONS

WO 96/22781: METHOD FOR PREPARING MACROPHAGES, AND KITS AND COMPOSITIONS THEREFOR

WO 97/44441: NEW ANTIGEN PRESENTING CELLS, A PROCESS FOR PREPARING THE SAME AND THEIR USE AS CELLULAR VACCINES

1. Dendritic cells irreversibly triggered to maturation, which are CD14 positive, which express MHC class I with a median fluorescence intensity less than about 1500 and CD86 with a median fluorescence intensity less than about 500, as determined by immunofluorescence staining and flow cytometry analysis.

2. Dendritic cells according to claim 1 which secrete less than about 3000 pg/ml of IL-12p70 and less than about 500 pg/ml of IL-10, as determined by Elisa assay for 10⁶ cells/ml.

3. Dendritic cells according to claim 1, which express MHC class I with a median fluorescence intensity less than about 700 and CD86 with a median fluorescence intensity less than about 300, as determined by immunofluorescence staining and flow cytometry analysis, and which secrete less than about 80 pg/ml of IL-12p70 and less than about 300 pg/ml of IL-10, as determined by Elisa assay for 10⁶ cells/ml.

4. Dendritic cells according to claim 1, which are CD83 negative.

5. Dendritic cells according to claim 3, presenting the following characteristics:
CD83 expression with a median fluorescence intensity from about 3 to about 20 CD 14 expression with a median fluorescence intensity from about 20 to about 100 MHC Class I expression with a median fluorescence intensity from about 400 to about 700 CD86 expression with a median fluorescence intensity from about 100 to about 300 as determined by immunofluorescence staining and flow cytometry analysis, and: IL-12p70 secretion of 1 to about 80 pg/ml IL-10 secretion of about 15 to about 300 pg/ml. as determined by ELISA assay, for a total of 10⁶ cells/ml.

6. Dendritic cells according to claim 1 which originate from immature dendritic cells derived from blood monocytes cultured for 1 to 16 hours, preferably 6 hours.

7. Dendritic cells according to claim 1, which have the properties that they can be arrested in their maturation process, and to resume maturation after this arrest when cultivated in appropriate conditions.

8. Dendritic cells according to claim 1 which have the properties of becoming mature in vitro in a culture medium containing no maturating factors and no cytokines, for a sufficient culture time.

9. Dendritic cells according to claim 1, which have the properties of becoming mature in vivo after injection to a patient.

10. Dendritic cells according to claim 1, which have been loaded with a drug, a nucleic acid or an antigen of interest, for example a tumoral antigen.

11. Dendritic cells according to claim 10 which have been loaded with lysates of tumor cells, in particular melanoma tumor cell lines.

12. Dendritic cells according to claim 1, which promote the development of T helper CD4+ T cells, and which activate cytotoxic CD8+ T lymphocytes specific for an antigen, after previous contact between said antigen and phagocyting dendritic cells.

13. Composition of dendritic cells according to claim 1, under frozen form in an appropriate cryopreservative medium.

14. Process for obtaining dendritic cells irreversibly triggered to maturation, which are CD14 positive, which express MHC class I with a median fluorescence intensity inferior to about 1500 and CD86 with a median fluorescence intensity inferior to about 500, as determined by immunofluorescence staining and flow cytometry analysis, comprising a step of contacting immature dendritic cells derived from blood monocytes and incubating them for 1 to 16 hours, preferably 6 h, with a combination of two factors: a—Cytokine or agonist of cytokine or cytokine inducing factor, and b—Bacterial mixture of membrane fractions and/or ribosomal fractions, or ligand or an agonist, said ligand or its agonist being different from a cytokine.

15. Process according to claim 14 for obtaining dendritic cells irreversibly triggered to maturation, which express MHC class I with a median fluorescence intensity inferior to about 700 and CD86 with a median fluorescence intensity inferior to about 300, as determined by immunofluorescence staining and flow cytometry analysis, and which secrete less than about 80 pg/ml of IL-12p70 and less than about 300 pg/ml of IL-10 as determined by Elisa assay for 10⁶ cells/ml.

16. Process according to claim 14 where obtained dendritic cells are CD83 negative.

17. Process according to claim 14, wherein the cytokine is IFNγ, or wherein the cytokine inducing factor is poly I:C.

18. Process according to claim 14 wherein the bacterial mixture of membrane fractions and/or ribosomal fractions is a membrane subtraction of one strain of bacteria.

19. Process according to claim 18 wherein the membrane subfraction is a purified protein obtained from said membrane subfraction.

20. Process according to claim 18, wherein the bacterial mixture of membrane and/or ribosomal fractions is Ribomunyl^{R}, and the membrane subtraction is FMKp (Klebsiella pneumoniae membrane fraction).

21. Process according to claim 14, wherein the cytokine is IFNγ and the membrane subfraction is FMKp.

22. Process according to claim 21 wherein the used concentration of IFNγ is about 500 U/ml and the used concentration of FMKp is about 1 μg/ml.

23. Process according to claim 14, wherein the ligand is an antibody anti CD40 or a CD40 ligand.

24. Process according to claim 14, wherein the ligand is an inducible Heat shock protein 70 or isolated polypeptide sequences from it.

25. Dendritic cells irreversibly triggered to maturation such as obtained by the process according to claim 14.

26. Mature dendritic cells which have a secretion of IL-12p70 higher than secretion of IL-10.

27. Mature dendritic cells which secrete more than about 1000 pg/ml of IL-12p70 and less than about 100 pg/ml of IL-10 (as determined by Elisa assay for 10⁶ cells/ml) for at least 24 hours, and stimulate Th1 and cytotoxic immune response.

28. Process for preparing mature dendritic cells from irreversibly triggered dendritic cells according to claim 1, which comprises a step of culture of said irreversibly triggered dendritic cells without exogenous maturation factor nor cytokine added, in vitro or in vivo.

29. Mature dendritic cells such as obtained by the process according to claim 27.

30. Pharmaceutical composition containing as active substance dendritic cells irreversibly triggered to maturation, according to claim 1, having interiorised antigens, preferably vaccinal antigens, in association with a pharmaceutically acceptable vehicle.

31. Cellular vaccine composition containing as active substance dendritic cells irreversibly triggered to maturation according to claim 1, in a amount of about 10⁴ to about 10⁹, and preferably about 10⁵ to about 10⁷ of said cells per vaccinal dose.
